(19) United States
(12) Patent Application Publication
(10) Pub. No.: US 2025/0017619 A1
(43) Pub. Date: Jan. 16, 2025

(54) VESSEL SCORING DEVICE

(71) Applicant: Boston Scientific Scimed, Inc., Maple Grove, MN (US)

(72) Inventors: Joel M. WasDyke, Independence, MN (US); James Hemerick, Brooklyn Park, MN (US)

(73) Assignee: Boston Scientific Scimed, Inc., Maple Grove, MN (US)

(21) Appl. No.: 18/769,873

(22) Filed: Jul. 11, 2024

Related U.S. Application Data
(60) Provisional application No. 63/526,766, filed on Jul. 14, 2023.

(51) Int. Cl. A61B 17/3207; A61B 17/00

(52) U.S. Cl. CPC A61B 17/320725; A61B 2017/00557

(57) ABSTRACT

A medical device includes a catheter shaft including a distal region and an inflatable balloon that is secured to the distal region of the catheter shaft. The inflatable balloon has an outer surface and defines a centerline when inflated. A blade extends over and is secured to the outer surface, the blade having a cutting edge extending from a distal end of the blade to a proximal end of the blade, when the inflatable balloon is inflated, the cutting edge extends to a maximum distance measured relative to the centerline of the inflatable balloon at the distal end of the blade and a minimum distance measured relative to the centerline of the inflatable balloon of the medical device at the proximal end of the blade.

### CROSS REFERENCE TO RELATED APPLICATIONS

This application claims the benefit of U.S. Provisional Patent Application Ser. No. 63/526,766, filed Jul. 14, 2023, which is incorporated herein by reference.

### TECHNICAL FIELD

The disclosure is directed to angioplasty balloon catheters including one or more cutting blades mounted to the balloon. More particularly, the disclosure is directed to cutting blades of a cutting balloon catheter that taper from a distal end to a proximal end.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action, or lack of oxygenation and/or circulation to other regions of the body.

Occluded, stenotic, or narrowed blood vessels, as well as native or synthetic arteriovenous dialysis fistulae, may be treated in a recanalization procedure, such as with an angioplasty balloon catheter advanced over a guidewire to an occlusion so that the balloon is positioned across the occlusion. The balloon is then inflated to enlarge the passageway through the occlusion.

One of the major obstacles in treating coronary artery disease and/or treating blocked blood vessels or fistulae is re-stenosis or re-narrowing of the passageway through the occlusion subsequent to an angioplasty procedure or other recanalization procedure. Evidence has shown that cracking, cutting or scoring the stenosis, for example, with an angioplasty balloon equipped with a blade member, during treatment can reduce incidence of re-stenosis. Additionally, cracking, cutting or scoring the stenosis may reduce trauma at the treatment site and/or may reduce the trauma to adjacent healthy tissue. Blade members may also be beneficial additions to angioplasty procedures when the targeted occlusion is hardened or calcified. It is believed typical angioplasty balloons, alone, may not be able to expand certain of these hardened lesions. Thus, angioplasty balloons equipped with blade members have been developed to attempt to enhance angioplasty treatments. Blade members may be adapted to cut, crack or score lesions, for example. Some lesions are longer than the blade members. There is an ongoing need for blade members that are adapted to treat long lesions while still permitting delivery through tortuous portions of the vasculature.

### SUMMARY

The disclosure is directed to several alternative designs, materials and methods of manufacturing medical device structures and assemblies, and the use thereof. An example may be found in a medical device. The medical device includes a catheter shaft including a distal region and an inflatable balloon secured to the distal region of the catheter shaft, the inflatable balloon having an outer surface, the inflatable balloon defining a centerline when inflated. A blade extends over and is secured to the outer surface, the blade having a cutting edge extending from a distal end of the blade to a proximal end of the blade, when the inflatable balloon is inflated, the cutting edge tapers from a maximum distance measured relative to a centerline of the balloon at the distal end of the blade to a minimum distance measured relative to the centerline of the balloon at the proximal end of the blade.

Alternatively or additionally, the cutting edge of the blade may include a straight line.

Alternatively or additionally, when inflated, the outer surface of the inflatable balloon may be a constant distance from the centerline of the inflatable balloon.

Alternatively or additionally, the cutting edge of the blade may taper from the distal end of the blade to the proximal end of the blade.

Alternatively or additionally, the cutting edge of the blade may define a blade height above the outer surface of the inflatable balloon, and the blade height may taper from a maximum blade height at the distal end of the blade to a minimum blade height at the proximal end of the blade.

Alternatively or additionally, when inflated, the outer surface of the inflatable balloon may be a varying distance relative to the centerline of the inflatable balloon.

Alternatively or additionally, the distance between the outer surface of the inflatable balloon and the longitudinal axis may be at a maximum at a distal region of the inflatable balloon and may be at a minimum at a proximal region of the inflatable balloon.

Alternatively or additionally, the cutting edge of the blade may have a constant height relative to the outer surface of the inflatable balloon.

Alternatively or additionally, the cutting edge of the blade may have a varying height relative to the outer surface of the inflatable balloon.

Alternatively or additionally, the medical device may further include one or more additional blades, each of the one or more additional blades extending over and secured to the outer surface, each of the one or more additional blades having a cutting edge extending from a distal end of the blade to a proximal end of the blade, the cutting edge extending when the inflatable balloon is inflated from a maximum distance measured relative to a centerline of the inflatable balloon at the distal end of the blade to a minimum distance measured relative to the centerline of the inflatable balloon at the proximal end of the blade.

Another example may be found in a medical device. The medical device includes a catheter shaft including a distal region and an inflatable balloon secured to the distal region of the catheter shaft. The inflatable balloon includes a constant diameter middle section corresponding to where the polymeric pad is secured to the outer surface of the inflatable balloon, a distal region that tapers to a distal waist, and a proximal region that tapers to a proximal waist. A polymeric pad is secured to the constant diameter middle section of the inflatable balloon. A blade is secured within the polymeric pad, the blade defining a tapered cutting edge extending from a distal end of the blade to a proximal end of the blade, the tapered cutting edge having a maximum blade height relative to the polymeric pad at the distal end of the blade and a minimum blade height relative to the polymeric pad at the proximal end of the blade.

Alternatively or additionally, the constant diameter middle section may have a diameter when inflated in a range of 1.5 to 2.5 millimeters.

Alternatively or additionally, the minimum blade height may be in a range of 0 to 0.5 millimeters and the maximum blade height may be in a range of 1 to 2 millimeters.

Another example may be found in a medical device. The medical device includes a catheter shaft including a distal region. An inflatable balloon is secured to the distal region of the catheter shaft and includes a tapered middle section corresponding to where the polymeric pad is secured to the outer surface of the inflatable balloon, the tapered middle section tapering from a maximum diameter near a distal region to a minimum diameter near a proximal region. A polymeric pad is secured to the tapered middle section of the inflatable balloon and a blade is secured within the polymeric pad, the blade defining a cutting edge extending from a distal end of the blade to a proximal end of the blade.

Alternatively or additionally, the cutting edge may have a uniform height measured relative to the polymeric pad.

Another example may be found in a medical device. The medical device includes a catheter shaft including a distal region, and an inflatable balloon secured to the distal region of the catheter shaft, the inflatable balloon having an outer surface, the inflatable balloon defining a centerline when inflated. A blade extends over and is secured to the outer surface, the blade having a cutting edge extending from a distal end of the blade to a proximal end of the blade, when the inflatable balloon is inflated, the cutting edge tapers from a maximum distance measured relative to a centerline of the balloon at the distal end of the blade to a minimum distance measured relative to the centerline of the balloon at the proximal end of the blade.

Alternatively or additionally, the cutting edge of the blade may include a straight line.

Alternatively or additionally, the minimum distance may be in a range of 1 to 2 millimeters and the maximum distance may be in a range of 2 to 4 millimeters.

Alternatively or additionally, the medical device may further include a braid extending through at least a portion of the elongate shaft and/or the inflatable balloon.

Alternatively or additionally, when inflated, the outer surface of the inflatable balloon may be a constant distance from the centerline of the balloon.

Alternatively or additionally, the cutting edge of the blade may taper from the distal end of the blade to the proximal end of the blade.

Alternatively or additionally, the cutting edge of the blade may define a blade height above the outer surface of the inflatable balloon, and the blade height may taper from a maximum blade height at the distal end of the blade to a minimum blade height at the proximal end of the blade.

Alternatively or additionally, the maximum blade height may be in a range of 1 to 2 millimeters.

Alternatively or additionally, the minimum blade height may be in a range of 0 to 0.5 millimeters.

Alternatively or additionally, when inflated, the outer surface of the inflatable balloon may be a varying distance relative to the centerline of the balloon.

Alternatively or additionally, the distance between the outer surface of the inflatable balloon and the longitudinal axis may be at a maximum at a distal region of the inflatable balloon and may be at a minimum at a proximal region of the inflatable balloon.

Alternatively or additionally, the cutting edge of the blade may have a constant height relative to the outer surface of the inflatable balloon.

Alternatively or additionally, the cutting edge of the blade may have a varying height relative to the outer surface of the inflatable balloon.

Alternatively or additionally, the medical device may further include one or more additional blades, each of the one or more additional blades extending over and secured to the outer surface, each of the one or more additional blades having a cutting edge extending from a distal end of the blade to a proximal end of the blade, the cutting edge extending when the inflatable balloon is inflated from a maximum distance measured relative to a centerline of the balloon at the distal end of the blade to a minimum distance measured relative to the centerline of the balloon at the proximal end of the blade.

Another example may be found in a medical device. The medical device includes a catheter shaft including a distal region. An inflatable balloon is secured to the distal region of the catheter shaft and includes a constant diameter middle section corresponding to where the polymeric pad is secured to the outer surface of the inflatable balloon, a distal region that tapers to a distal waist, and a proximal region that tapers to a proximal waist. A polymeric pad is secured to the constant diameter middle section of the inflatable balloon. A blade is secured within the polymeric pad, the blade defining a tapered cutting edge extending from a distal end of the blade to a proximal end of the blade, the tapered cutting edge having a maximum blade height relative to the polymeric pad at the distal end of the blade and a minimum blade height relative to the polymeric pad at the proximal end of the blade.

Alternatively or additionally, the constant diameter middle section may have a diameter when inflated in a range of 1.5 to 2.5 millimeters.

Alternatively or additionally, the minimum blade height may be in a range of 0 to 0.5 millimeters and the maximum blade height may be in a range of 1 to 2 millimeters.

Another example may be found in a medical device. The medical device includes a catheter shaft including a distal region. An inflatable balloon is secured to the distal region of the catheter shaft, the inflatable balloon including a tapered middle section corresponding to where the polymeric pad is secured to the outer surface of the inflatable balloon, the tapered middle section tapering from a maximum diameter near a distal region to a minimum diameter near a proximal region. A polymeric pad is secured to the tapered middle section of the inflatable balloon. A blade is secured within the polymeric pad, the blade defining a cutting edge extending from a distal end of the blade to a proximal end of the blade.

Alternatively or additionally, the cutting edge may have a uniform height measured relative to the polymeric pad.

Alternatively or additionally, the cutting edge may have a varying height measured relative to the polymeric pad.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic partially cross-sectioned view of an example medical device;

FIG. 2 is a schematic view of an example medical device;

FIG. 3 is a schematic view of an example medical device;

FIG. 4 is a schematic view of an example medical device;

FIG. 5 is a schematic view of an example medical device;

FIG. 6 is a schematic view of an example medical device;

FIG. 7 is a schematic view of an example medical device;

FIG. 8 is a schematic view of an example medical device;

FIG. 9 is a schematic view of an example medical device;

FIGS. 10 through 14 are schematic views of an example medical device deployed within the vasculature proximate a lesion;

FIG. 15 is a schematic view of an example medical device;

FIG. 16 is a cross-sectional view taken along the line 16-16 of FIG. 15;

FIG. 17 is a cross-sectional view taken along the line 17-17 of FIG. 15;

FIG. 18 is a schematic view of an example medical device;

FIG. 19 is a cross-sectional view taken along the line 19-19 of FIG. 18;

FIG. 20 is a cross-sectional view taken along the line 20-20 of FIG. 18; and

FIG. 21 is a cross-sectional view taken along the line 21-21 of FIG. 20.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

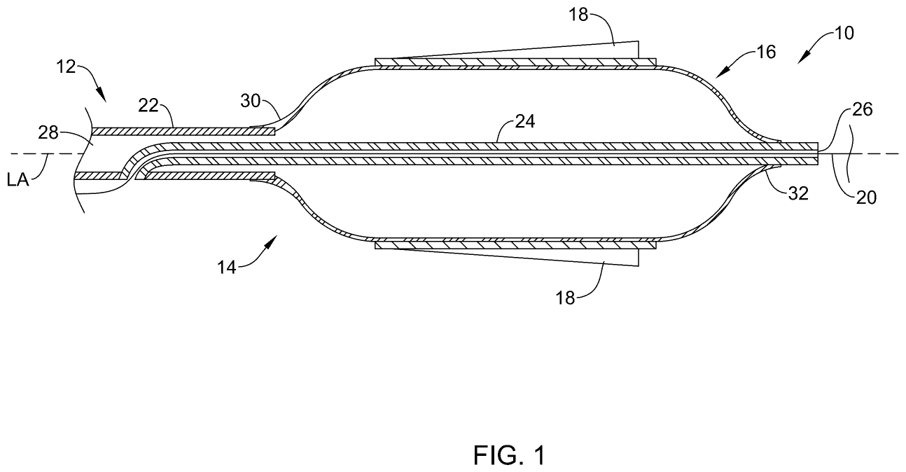

FIG. 1 is a partial cross-sectional side view of an example medical device 10. In some instances, the example medical device 10 may be utilized within a patient's vasculature in order to treat a stenosis within the vasculature. In some instances, the medical device 10 may be used to crack or cut a calcium lesion. The medical device 10 may define a longitudinal axis “LA”, as shown. The medical device 10 includes a catheter shaft 12 having a distal region 14. An inflatable balloon 16 may be secured to the distal region 14 of the catheter shaft 12. One or more blades 18 may be mounted on or otherwise secured relative to the inflatable balloon 16. In some instances, the one or more blades 18 may be secured to the inflatable balloon 16 via a polymeric pad 19. In some instances, as will be described with respect to FIGS. 3 through 7, each of the one or more blades 18 may include a base portion that is at least partially embedded within the polymeric pad 19 and a blade portion that forms the visible portion of the blade 18. The polymeric pad 19 may be adhesively secured to the inflatable balloon 16, for example.

In some instances, the medical device 10 may be advanced over a guidewire 20, through the vasculature, to a target area. Once positioned at the target location in the vasculature, the inflatable balloon 16 can be inflated to exert a radially outward force on a lesion, as the blades 18 engage the lesion. Thus, the blades 18 may crack, cut or score the lesion to facilitate enlarging the lumen proximate the lesion. The target area may be within any suitable peripheral or cardiac vessel lumen location. In some instances, the medical device 10 may be withdrawn proximally with the inflatable balloon 16 inflated in order to crack, cut or score the lesion, particularly in circumstances in which the lesion is longer than the blades 18.

The blades 18 may vary in number, position, and arrangement about the inflatable balloon 16. For example, the medical device 10 may include one, two, three, four, five, six, or more blades 18 that are disposed at any position along the inflatable balloon 16 and in a regular, irregular, or any other suitable pattern. For example, in some instances, the inflatable balloon 16 may include a plurality of blades 18 longitudinally arranged symmetrically around a circumference of the inflatable balloon 16.

The blades 18 may be made from any suitable material such as a metal, metal alloy, polymer, metal-polymer composite, and the like, or any other suitable material. For example, the blades 18 may be made from stainless steel, titanium, nickel-titanium alloys, tantalum, iron-cobalt-nickel alloys, or other metallic materials in some instances. The blades 18 may have a triangular cross-sectional shape, or a square cross-sectional shape, or a rectangular cross-sectional shape, a circular cross-sectional shape or any other desired configuration. In some instances, the blades 18 may be tapered.

The blades 18 may be mounted to the inflatable balloon 16 using a variety of techniques. In some cases, each of the blades 18 may include a base portion that includes cutouts for flexibility, where the base portion may be at least partially embedded in a polymeric member (not shown) that may itself be secured to an outer surface of the inflatable balloon 16. Additional details regarding how the blades 18 may be secured to the outer surface of the inflatable balloon 16, including the aforementioned polymeric member, are described for example in U.S. Pat. Nos. 9,226,768; 10,046,146; 10,058,349; and 10,729,893, which are incorporated by reference in their entirety.

The inflatable balloon 16 may be made from angioplasty balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. In some instances, the inflatable balloon 16 may include a single layer of material, whereas in other instances the inflatable balloon 16 may be of a multi-layer construction, including a plurality of layers of materials. For instance, the inflatable balloon 16 may be formed as a co-extrusion or tri-layer extrusion in some instances. In some instances, the inflatable balloon 16 may be adapted to be compliant, and may be inflated at a relatively low pressure such as 2 to 4 atmospheres.

The inflatable balloon 16 may be configured so that the inflatable balloon 16 includes one or more “wings” or wing-shaped regions when the inflatable balloon 16 is deflated. In some instances, the wings may be configured so that the blade members 18 can be positioned at the inward-most positions of the deflated inflatable balloon 16, with the wings of the balloon folds positioned between adjacent blade members 18. This arrangement may reduce the exposure of the blade members 18 to the blood vessel during delivery of the inflatable balloon 16 to the lesion.

The catheter shaft 12 may be a catheter shaft, similar to typical catheter shafts. For example, the catheter shaft 12 may include an outer tubular member 22 and an inner tubular member 24 extending through at least a portion of the outer tubular member 22. The outer tubular member 22 and the inner tubular member 24 may each be manufactured from a number of different materials. For example, the outer tubular member 22 and the inner tubular member 24 may be made of metals, metal alloys, polymers, metal-polymer composites or any other suitable materials.

The outer tubular member 22 and the inner tubular member 24 may be arranged in any appropriate way. For example, in some embodiments the inner tubular member 24 can be disposed coaxially within the outer tubular member 22. According to these embodiments, the inner tubular member 24 and the outer tubular member 22 may or may not be secured to one another along the general longitudinal axis of the catheter shaft 12. Alternatively, the inner tubular member 24 may follow the inner wall or otherwise be disposed adjacent the inner wall of the outer tubular member 22. In other embodiments, the outer tubular member 22 and the inner tubular member 24 may be arranged in another desired fashion.

The inner tubular member 24 may include an inner lumen 26. In at least some instances, the inner lumen 26 is a guidewire lumen for receiving the guidewire 20 therethrough. Accordingly, the medical device 10 can be advanced over the guidewire 20 to the desired location. The guidewire lumen 26 may extend along essentially the entire length of the catheter shaft 12 such that the medical device 10 resembles a traditional “over-the-wire” catheter. Alternatively, and as shown, the guidewire lumen 26 may extend along only a portion of the catheter shaft 12 such that the medical device 10 resembles a “single-operator-exchange” or “rapid-exchange” catheter.

The catheter shaft 12 may also include an inflation lumen 28 that may be used, for example, to transport inflation media to and from the inflatable balloon 16 to selectively inflate and/or deflate the inflatable balloon 16. The location and position of the inflation lumen 28 may vary, depending on the configuration of the outer tubular member 22 and the inner tubular member 24. For example, when the outer tubular member 22 surrounds the inner tubular member 24, the inflation lumen 28 may be defined within the space between the outer tubular member 22 and the inner tubular member 24. In instances in which the outer tubular member 22 is disposed alongside the inner tubular member 24, then the inflation lumen 28 may be the lumen of the outer tubular member 22.

The inflatable balloon 16 may be coupled to the catheter shaft 12 in any of a number of suitable ways. For example, the inflatable balloon 16 may be adhesively or thermally bonded to the catheter shaft 12. In some embodiments, a proximal waist 30 of the inflatable balloon 16 may be bonded to the catheter shaft 12, for example, bonded to a distal end of the outer tubular member 22, and a distal waist 32 of the inflatable balloon 16 may be bonded to the catheter shaft 12, for example, bonded to a distal end of the inner tubular member 24. The exact bonding positions, however, may vary.

In some instances, as noted, the medical device 10 may be adapted to be pulled through a lesion in order to treat a lesion that may be longer than the blades 18. While in theory the blades 18 may be made any length, it will be appreciated that longer blades may be more difficult to deliver, particularly through a tortuous vasculature. In some instances, in order to be more readily pulled proximally through a lesion, the blades 18 may be adapted to present a tapered cutting edge to the lesion. In some instances, a tapered blade may be secured to a constant diameter balloon, with the taper in the blade providing an overall taper in a cutting edge exposed to the lesion. In some instances, a constant height blade may be secured to a variable diameter balloon, with the variable diameter balloon providing an overall taper in a cutting edge exposed to the lesion. In some instances, a tapered blade may be secured to a variable diameter balloon, such that both the tapered blade and the variable diameter balloon contribute to an overall taper in a cutting edge exposed to the lesion.

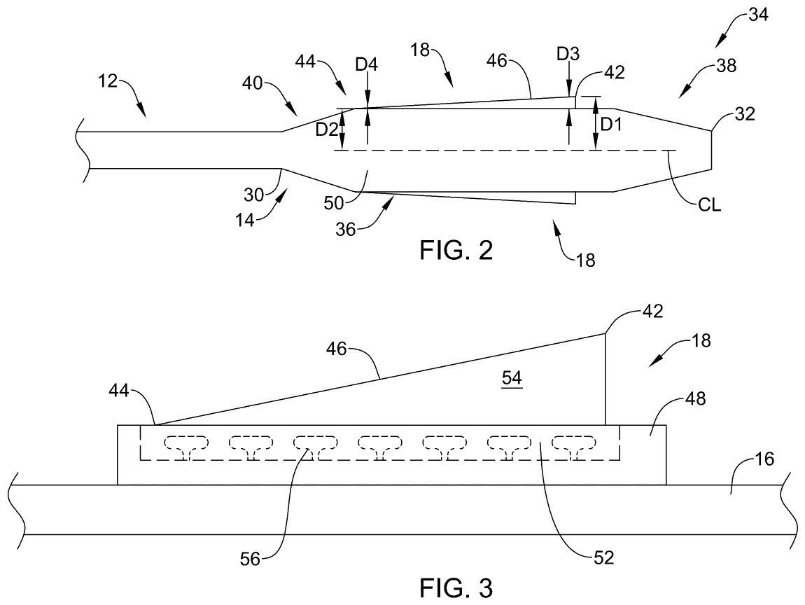

FIG. 2 is a schematic view of an example medical device 34. The example medical device 34 may be considered as being an example of the medical device 10. The medical device 34 includes the inflatable balloon 16 secured relative to the distal region 14 of the elongate shaft 12. The inflatable balloon 16 can be seen as including a constant diameter middle section 36, a distal region 38 that tapers from the constant diameter middle section 36 to the distal waist 32, and a proximal region 40 that tapers from the constant diameter middle section 36 to the proximal waist 30. In some instances, the medical device 34 includes one or more tapered blades 18. The tapered blades 18 taper from a distal end 42 to a proximal end 44, and present a tapered cutting edge 46 that extends between the distal end 42 and the proximal end 44. In some instances, the tapered cutting edge 46 adapts the medical device 34 to be pulled proximally through a lesion with the inflatable balloon 16 inflated. The tapered cutting edge 46 may be considered as defining a straight line.

Having the inflatable balloon 16 inflated pushes the tapered cutting edge 46 into a position in which the tapered cutting edge 46 is able to cut or score the lesion as the medical device 34 is pulled proximally through the lesion. In some instances, the inflatable balloon 16 may be dimensioned to push the tapered cutting edge 46 into a position to contact the lesion but is not large enough for the inflatable balloon 16 itself to crack or otherwise deform the lesion. As an example, the inflatable balloon 16 may have a diameter (within the constant diameter middle section 36) in a range of 1.5 millimeters to 2.5 millimeters, although other sizes are contemplated.

The inflatable balloon 16 may be considered as having a centerline CL that extends axially through the inflatable balloon 16. In some instances, the centerline CL may be considered as being colinear with the longitudinal axis LA shown in FIG. 1. In some instances, the tapered cutting edge 46 may be measured with respect to distance from the centerline CL. As an example, the tapered cutting edge 46 may taper from a maximum distance D₁ measured radially between the distal end 42 of the tapered blade 18 and the centerline CL to a minimum distance D₂ measured radially between the proximal end 44 of the tapered blade 18 and the centerline CL. In some instances, the maximum distance D₁ may be in a range of 2 millimeters to 4 millimeters while the minimum distance D₂ may be in a range of 1 millimeter to 2 millimeters. In some instances, the tapered blade 18 may itself have a blade height that is measured relative to an outer surface 50 of the inflatable balloon 16, with a maximum blade height at the distal end 42 of the tapered blade 18, indicated as D₃. The tapered blade 18 may have a minimum blade height at the proximal end 44 of the tapered blade 18, indicated as D₄, that is in a range of 0 to 0.5 millimeters.

In some instances, the tapered blade 18 may be secured directly to the outer surface of the inflatable balloon 16. In some instances, as seen for example in FIG. 3, the tapered blade 18 may be secured within a polymeric pad 48. The tapered blade 18 may be considered as including a base portion 52 that is embedded within the polymeric pad 48 and a blade portion 54 that extends outwardly from the base portion 52. In some instances, the base portion 52 may be slightly longer than the blade portion 54. In some instances, the blade portion 54 may be considered as defining the blade heights D₃ and D₄. In some instances, the base portion 52 may include cutouts for flexibility, where the base portion 52 may be at least partially embedded in the polymeric pad 48 that may itself be secured to an outer surface of the inflatable balloon 16. In some instances, the base portion 52 may include T-shaped slots 56 that are adapted to provide additional flexibility to the base portion 52 as well as to help embed the base portion 52 within the polymeric pad 48. Additional details regarding how the blade 18 may be secured to the outer surface of the inflatable balloon 16, including the aforementioned polymeric member, are described for example in U.S. Pat. Nos. 9,226,768; 10,046,146; 10,058,349; and 10,729,893, which are incorporated by reference in their entirety.

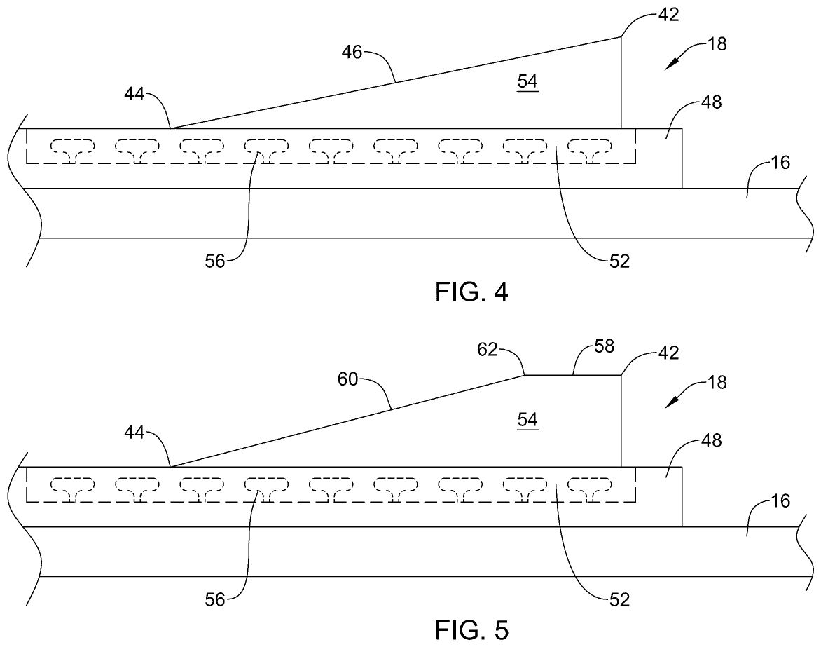

FIG. 4 shows the blade 18 may be secured within the polymeric pad 48, with the base portion 52 of the blade 18 embedded within the polymeric pad 48 and the blade portion 54 extending outwardly from the base portion 52. In some instances, as shown, the base portion 52 may extend proximally a distance from the proximal end 44 of the blade 18. In some instances, having the base portion 52 extend in this manner provides additional securement for the blade 18 within the polymeric pad 48. In some instances, the base portion 52 may include T-shaped slots 56 that are adapted to provide additional flexibility to the base portion 52 as well as to help embed the base portion 52 within the polymeric pad 48. In FIG. 4, the blade 18 includes the tapered cutting edge 46 that tapers linearly from the distal end 42 of the blade 18 to the proximal end 44 of the blade 18. In some instances, the tapered cutting edge 46 facilitates pulling the blade 18 proximally through a lesion with the inflatable balloon 16 inflated. This may be useful, particularly when the lesion is longer than the blade or blades 18.

FIG. 5 shows the blade 18 may be secured within the polymeric pad 48, with the base portion 52 of the blade 18 embedded within the polymeric pad 48 and the blade portion 54 extending outwardly from the base portion 52. In some instances, as shown, the base portion 52 may extend proximally a distance from the proximal end 44 of the blade 18. In some instances, having the base portion 52 extend in this manner provides additional securement for the blade 18 within the polymeric pad 48. In some instances, the base portion 52 may include T-shaped slots 56 that are adapted to provide additional flexibility to the base portion 52 as well as to help embed the base portion 52 within the polymeric pad 48.

In FIG. 5, the blade 18 includes a first cutting edge portion 58 and a second cutting edge portion 60. As shown, the first cutting edge portion 58 may be considered as being parallel or at least substantially parallel with the polymeric pad 48, where substantially parallel is defined as being within ten percent of parallel. The first cutting edge portion 58 extends between an inflection point 62 and the distal end 42 of the blade 18. The second cutting edge portion 60 tapers proximally from the inflection point 62 to the proximal end 44 of the blade 18. In some instances, the second cutting edge portion 60 facilitates pulling the blade 18 proximally through a lesion with the inflatable balloon 16 inflated. This may be useful, particularly when the lesion is longer than the blade or blades 18.

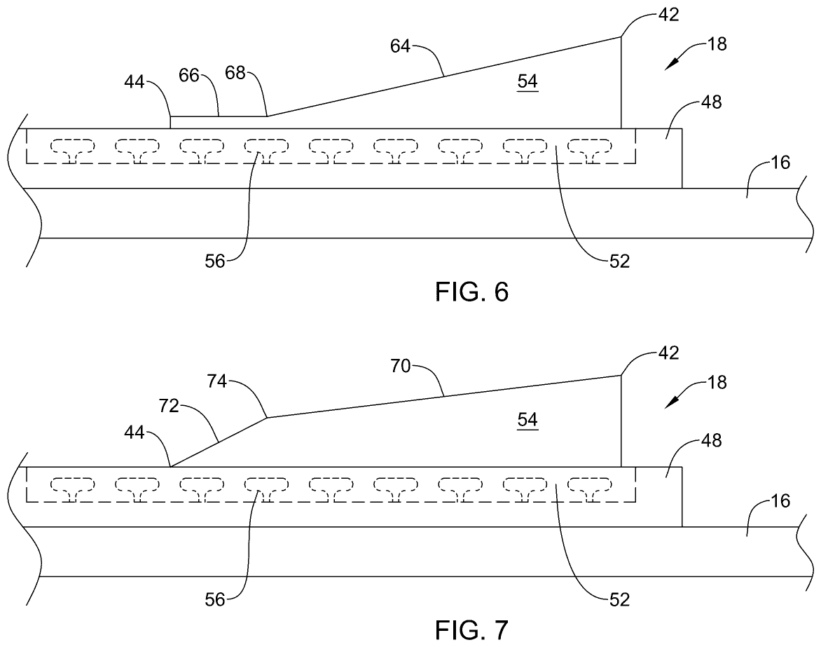

FIG. 6 shows the blade 18 may be secured within the polymeric pad 48, with the base portion 52 of the blade 18 embedded within the polymeric pad 48 and the blade portion 54 extending outwardly from the base portion 52. In some instances, as shown, the base portion 52 may extend proximally a distance from the proximal end 44 of the blade 18. In some instances, having the base portion 52 extend in this manner provides additional securement for the blade 18 within the polymeric pad 48. In some instances, the base portion 52 may include T-shaped slots 56 that are adapted to provide additional flexibility to the base portion 52 as well as to help embed the base portion 52 within the polymeric pad 48.

In FIG. 6, the blade 18 includes a first cutting edge portion 64 and a second cutting edge portion 66. As shown, the first cutting edge portion 64 may be considered as tapering distally from the distal end 42 of the blade 18 to an inflection point 68 that represents an intersection of the first cutting edge portion 64 and the second cutting edge portion 66. The second cutting edge portion 66 may be considered as being parallel or at least substantially parallel with the polymeric pad 48, where substantially parallel is defined as being within ten percent of parallel. The second cutting edge portion 66 extends from the inflection point 68 to the proximal end 44 of the blade 18. In some instances, the first cutting edge portion 64 and the second cutting edge portion 66 facilitate pulling the blade 18 proximally through a lesion with the inflatable balloon 16 inflated. This may be useful, particularly when the lesion is longer than the blade or blades 18.

FIG. 7 shows the blade 18 may be secured within the polymeric pad 48, with the base portion 52 of the blade 18 embedded within the polymeric pad 48 and the blade portion 54 extending outwardly from the base portion 52. In some instances, as shown, the base portion 52 may extend proximally a distance from the proximal end 44 of the blade 18. In some instances, having the base portion 52 extend in this manner provides additional securement for the blade 18 within the polymeric pad 48. In some instances, the base portion 52 may include T-shaped slots 56 that are adapted to provide additional flexibility to the base portion 52 as well as to help embed the base portion 52 within the polymeric pad 48.

In FIG. 7, the blade 18 includes a first cutting edge portion 70 and a second cutting edge portion 72. The first cutting edge portion 70 tapers proximally from the distal end 42 of the blade 18 to an inflection point 74. The second cutting edge portion 72 tapers proximally, albeit at a different angle, from the inflection point 74 to the proximal end 44 of the blade 18. In some instances, the first cutting edge portion 70 and the second cutting edge portion 72 facilitate pulling the blade 18 proximally through a lesion with the inflatable balloon 16 inflated. This may be useful, particularly when the lesion is longer than the blade or blades 18.

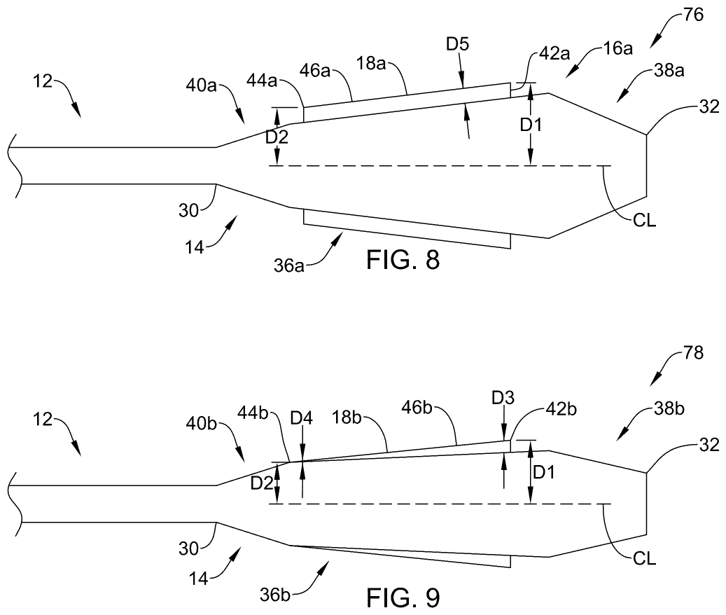

FIG. 8 is a schematic view of an example medical device 76. The example medical device 76 may be considered as being an example of the medical device 10. The medical device 76 includes an inflatable balloon 16a that is secured relative to the distal region 14 of the elongate shaft 12. The inflatable balloon 16a can be seen as including a varying diameter middle section 36a, a distal region 38a that tapers from the varying diameter middle section 36a to the distal waist 32, and a proximal region 40a that tapers from the varying diameter middle section 36a to the proximal waist 30.

The inflatable balloon 16a may be made from angioplasty balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. In some instances, the inflatable balloon 16a may include a single layer of material, whereas in other instances the inflatable balloon 16a may be of a multi-layer construction, including a plurality of layers of materials. For instance, the inflatable balloon 16a may be formed as a co-extrusion or tri-layer extrusion in some instances.

In some instances, the medical device 76 includes one or more blades 18a. The blades 18a have a constant blade height indicated as D₅. The blades 18a extend from a distal end 42 to a proximal end 44a, and present a tapered cutting edge 46a that extends between the distal end 42a and the proximal end 44a. In some instances, the tapered cutting edge 46a adapts the medical device 76 to be pulled proximally through a lesion with the inflatable balloon 16a inflated. The inflatable balloon 16a may be configured so that the inflatable balloon 16a includes one or more “wings” or wing-shaped regions when the inflatable balloon 16a is deflated. In some instances, the wings may be configured so that the blades 18a can be positioned at the inward-most positions of the deflated inflatable balloon 16a, with the wings of the balloon folds positioned between adjacent blades 18a. This arrangement may reduce the exposure of the blades 18a to the blood vessel during delivery of the inflatable balloon 16a to the lesion.

The blades 18a may be made from any suitable material such as a metal, metal alloy, polymer, metal-polymer composite, and the like, or any other suitable material. For example, the blades 18a may be made from stainless steel, titanium, nickel-titanium alloys, tantalum, iron-cobalt-nickel alloys, or other metallic materials in some instances. The blades 18a may have a triangular cross-sectional shape, or a square cross-sectional shape, or a rectangular cross-sectional shape, a circular cross-sectional shape or any other desired configuration.

Having the inflatable balloon 16a inflated pushes the tapered cutting edge 46a into a position in which the tapered cutting edge 46a is able to cut or score the lesion as the medical device 76 is pulled proximally through the lesion. In some instances, the inflatable balloon 16a may be dimensioned to push the tapered cutting edge 46a into a position to contact the lesion but is not large enough for the inflatable balloon 16a itself to crack or otherwise deform the lesion. As an example, the inflatable balloon 16a may have a maximum diameter in a range of 1.5 millimeters to 2.5 millimeters, although other sizes are contemplated. The inflatable balloon 16a may be considered as having a centerline CL that extends axially through the inflatable balloon 16a. In some instances, the centerline CL may be considered as being colinear with the longitudinal axis LA shown in FIG. 1. In some instances, the tapered cutting edge 46a may be measured with respect to distance from the centerline CL. As an example, the tapered cutting edge 46a may taper from a maximum distance D₁ measured radially between the distal end 42a of the blade 18a and the centerline CL to a minimum distance D₂ measured radially between the proximal end 44a of the blade 18a and the centerline CL. In some instances, the maximum distance D₁ may be in a range of 2 millimeters to 4 millimeters while the minimum distance D₂ may be in a range of 1 millimeter to 2 millimeters.

FIG. 9 is a schematic view of an example medical device 78. The example medical device 78 may be considered as being an example of the medical device 10. The medical device 78 includes an inflatable balloon 16b secured relative to the distal region 14 of the elongate shaft 12. The inflatable balloon 16b can be seen as including a varying diameter middle section 36b, a distal region 38b that tapers from the varying diameter middle section 36b to the distal waist 32, and a proximal region 40b that tapers from the varying diameter middle section 36b to the proximal waist 30.

The inflatable balloon 16b may be made from angioplasty balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. In some instances, the inflatable balloon 16b may include a single layer of material, whereas in other instances the inflatable balloon 16b may be of a multi-layer construction, including a plurality of layers of materials. For instance, the inflatable balloon 16b may be formed as a co-extrusion or tri-layer extrusion in some instances.

In some instances, the medical device 78 includes one or more tapered blades 18b. The tapered blades 18b taper from a distal end 42b to a proximal end 44b, and present a tapered cutting edge 46b that extends between the distal end 42b and the proximal end 44b. It will be appreciated that in FIG. 9, the tapered cutting edge 46b adapts the medical device 58 to be pulled proximally through a lesion with the inflatable balloon 16b inflated. Having the inflatable balloon 16b inflated pushes the tapered cutting edge 46b into a position in which the tapered cutting edge 46b is able to cut or score the lesion as the medical device 78 is pulled proximally through the lesion. In some instances, the inflatable balloon 16b may be dimensioned to push the tapered cutting edge 46b into a position to contact the lesion but is not large enough for the inflatable balloon 16b itself to crack or otherwise deform the lesion. As an example, the inflatable balloon 16b may have a maximum diameter in a range of 1.5 millimeters to 2.5 millimeters, although other sizes are contemplated.

The blades 18b may be made from any suitable material such as a metal, metal alloy, polymer, metal-polymer composite, and the like, or any other suitable material. For example, the blades 18b may be made from stainless steel, titanium, nickel-titanium alloys, tantalum, iron-cobalt-nickel alloys, or other metallic materials in some instances. The blades 18b may have a triangular cross-sectional shape, or a square cross-sectional shape, or a rectangular cross-sectional shape, a circular cross-sectional shape or any other desired configuration.

The inflatable balloon 16b may be considered as having a centerline CL that extends axially through the inflatable balloon 16b. In some instances, the centerline CL may be considered as being colinear with the longitudinal axis LA shown in FIG. 1. In some instances, the tapered cutting edge 46b may be measured with respect to distance from the centerline CL. As an example, the tapered cutting edge 46b may taper from a maximum distance D₁ measured radially between the distal end 42b of the tapered blade 18b and the centerline CL to a minimum distance D₂ measured radially between the proximal end 44b of the tapered blade 18b and the centerline CL. In some instances, the maximum distance D₁ may be in a range of 2 millimeters to 4 millimeters while the minimum distance D₂ may be in a range of 1 millimeter to 2 millimeters. In some instances, the tapered blade 18 may itself have a blade height that is measured relative to an outer surface 50 of the inflatable balloon 16, with a maximum blade height at the distal end 42b of the tapered blade 18b, indicated as D₃. The tapered blade 18 may have a minimum blade height at the proximal end 44b of the tapered blade 18b, indicated as D₄, that is in a range of 0 to 0.5 millimeters.

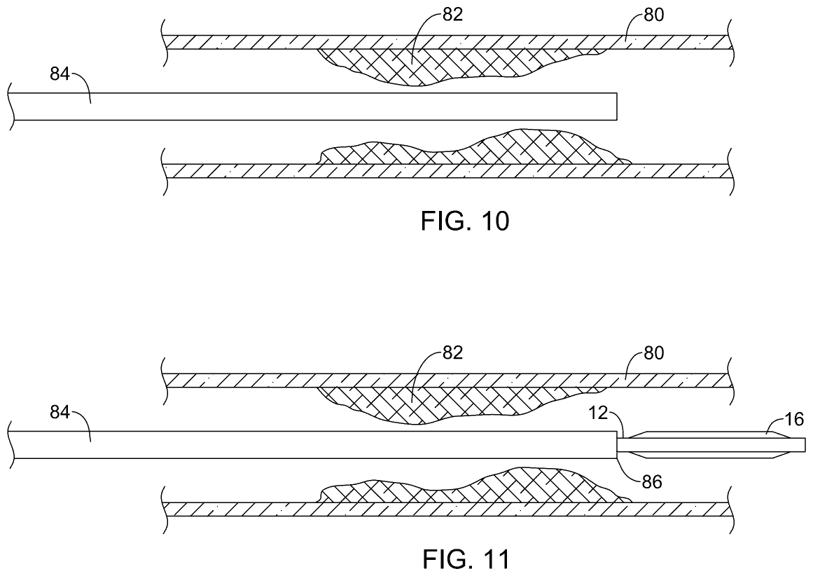

FIGS. 10 through 14 provide a schematic view of an example use of the medical device 10. While FIGS. 10 through 14 demonstrate use of the medical device 10, it will be appreciated that the medical device medical device 34 (with constant diameter balloon 16 and tapered blade 18), the medical device 76 (with tapered balloon 16a and constant height blade 18a) or the medical device 78 (with tapered balloon 16b and tapered blade 18b) may easily be used in the same manner. The various blade configurations shown in FIGS. 4 through 7 may also easily be used in the same manner.

In FIG. 10, a catheter 84 has been advanced through a blood vessel 80 to a position proximate a lesion 82. The lesion 82 may be a calcified lesion, for example. In some instances, the lesion 82 may be a fibrous lesion, particularly if the lesion 82 is located within the patient's venous system. In some instances, the catheter 84 may be advanced over a guidewire such as the guidewire 20 (not shown in FIG. 10). In some instances, the catheter 84 may be advanced without a guidewire.

In FIG. 11, it can be seen that the medical device 10 has been advanced through the catheter 84 and has begun to advance beyond a distal end 86 of the catheter 84. In some instances, the medical device 10 may be advanced through the catheter 84 without a guidewire. In some instances, the medical device 10 may be advanced over a guidewire such as the guidewire 20 extending through the catheter 84. In some instances, the medical device 10 may be advanced over a guidewire such as the guidewire 20 to a position as shown in FIG. 11 without use of the catheter 84. Once the medical device 10 has reached a position such as shown in FIG. 11, with the inflatable balloon 16 disposed distal of the lesion 82, the catheter 84 (if used) may be withdrawn.

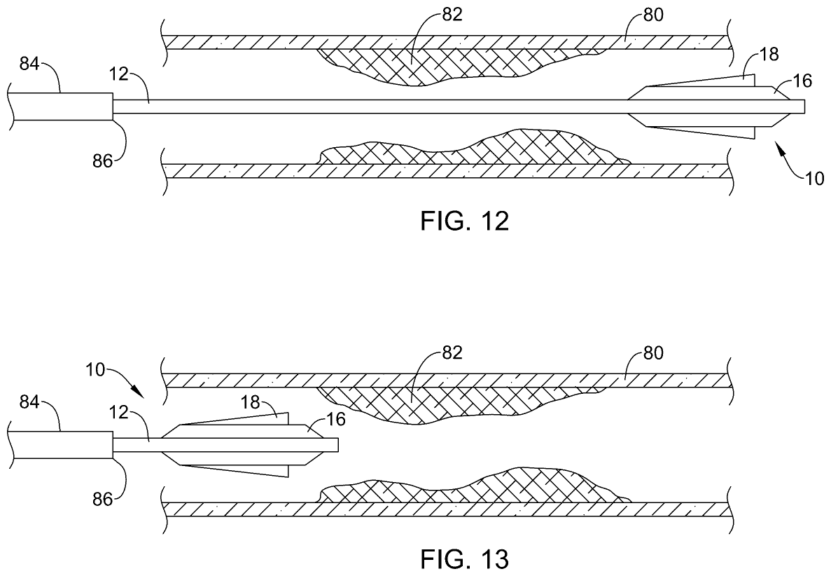

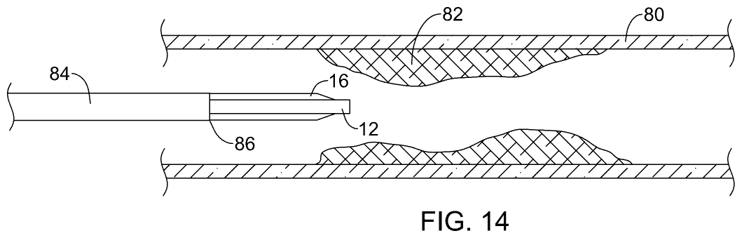

In FIG. 12, the catheter 84 has been withdrawn proximally, and no longer extends distally through the lesion 82. The inflatable balloon 16 has been inflated, which pushes the blades 18 in a radially outward direction. The elongate shaft 12 may be pulled proximally, thereby pulling the inflatable balloon 16 and the blades 18 proximally through the lesion 82. As the inflatable balloon 16 and the blades 18 move proximally through the lesion 82, the blades 18 score the lesion 82. The inflatable balloon 16 and the blades 18 will end up proximal of the lesion 82, as shown in FIG. 13. Subsequently, the inflatable balloon 16 may be deflated and the medical device 10 may be withdrawn back into the catheter 84 by either pushing the catheter 84 distally relative to the medical device 10, or by pulling the medical device 10 proximally in order to urge the medical device 10 back into the catheter 84, as shown in FIG. 14. The catheter 84 (and medical device 10) may be rotated before once again advancing the catheter 84 (and medical device 10) distally past the lesion 82 (as originally shown in FIGS. 10 and 11), and the process may be repeated (as shown in FIGS. 12 and 13) to provide additional cuts or scores on the lesion 82.

In some instances, the blade or blades 18 and/or the elongate shaft 12 and/or the inflatable balloon 16 may include an elutable drug coating. In some instances, a drug composition may be injected or otherwise inserted into the lumen extending through the elongate shaft 12, or the catheter 84, in order to provide a fluid drug composition in the area proximate the lesion 82. In some instances, forming a plurality of cuts or cracks within the lesion 82, such as using the process shown in FIGS. 10 through 14, may facilitate improving contact between the drug and an interior of the lesion 82. This may improve the efficacy of the drug, for example. A variety of different drugs are contemplated, including those that may help soften or dissolve the lesion 82.

In some instances, depending on the composition of the lesion 82, especially if the lesion 82 is within the arterial system, the blade or blades 18 may be able to cause cracks to form within the lesion 82. In some instances, depending on the composition of the lesion 82, especially if the lesion 82 is within the venous system, the blade or blades 18 may be able to cut into the lesion 82. In some instances, lesions within the venous system may tend to be softer and more fibrous, and thus do not crack as the more calcified lesions within the arterial system. It will be appreciated that formation of cracks or cuts within the lesion 82 may make it easier for a drug such as an elutable drug to penetrate further into the lesion 82.

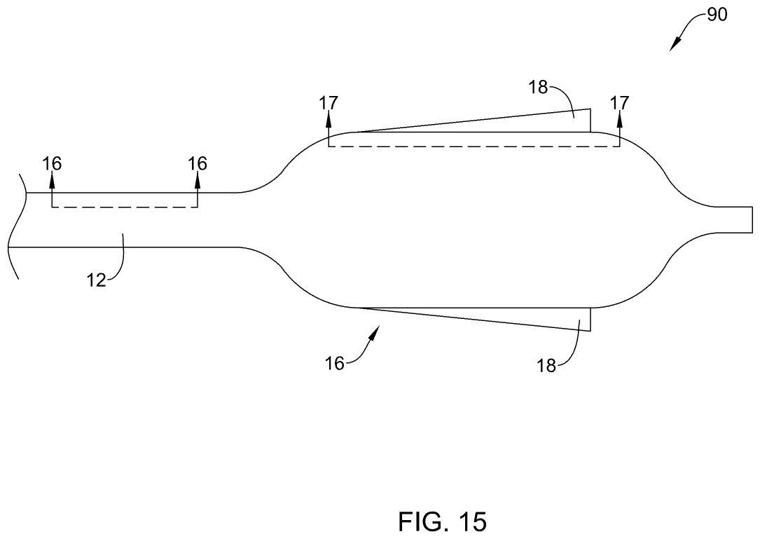

In some instances, the catheter shaft 12 and/or the inflatable balloon 16 may include one or more braids that may improve the tensile strength of the catheter shaft 12 and/or the inflatable balloon 16, particularly as the catheter shaft 12 is pulled proximally in order to pull the blade or blades 18 through the lesion 82. In some instances, a braid may extend at least partially over the blade or blades 18, and in some cases may help to secure the blade or blades 18 in place relative to the inflatable balloon 16. FIGS. 15 through 20 provide several examples of how one or more braids may be used within the catheter shaft 12 and/or the inflatable balloon 16. FIG. 15 is a schematic view of an example medical device 90. The example medical device 90 includes the inflatable balloon 16 secured to the elongate shaft 12. Two blades 18 are visible secured relative to the inflatable balloon 16. In some instances, there may be only one blade 18 secured relative to the inflatable balloon 16. In some instances, there may be three, four, five or more blades 18 secured relative to the inflatable balloon 16. In some instances, if there are two or more blades 18 secured relative to the inflatable balloon 16, the two or more blades 18 may be evenly spaced circumferentially about the inflatable balloon 16, or the blades 18 may be unevenly spaced circumferentially about the inflatable balloon 16.

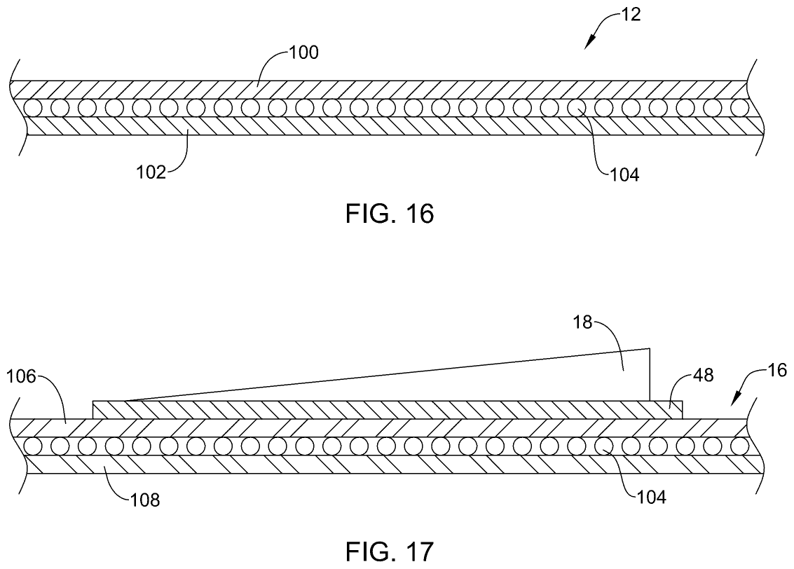

FIG. 16 is a cross-sectional view taken along the line 16-16 of FIG. 15, showing a cross-section through one wall of the elongate shaft 12. In some instances, the elongate shaft 12 may include an outer polymeric layer 100 and an inner polymeric layer 102. Each of the outer polymeric layer 100 and the inner polymeric layer 102 may be formed of any of a variety of different polymers including those listed below. In some instances, the inner polymeric layer 102 may be formed of a lubricious polymer such as a fluoropolymer. Polytetrafluoroethylene (PTFE) is an example of a fluoropolymer that may be used in forming the inner polymeric layer 102. In some instances, the inner polymeric layer 102 may not be formed of a lubricious polymer, but may include a lubricious coating. In some instances, the outer polymeric layer 100 may actually include two or more different polymeric layers. In some instances, the inner polymeric layer 102 may actually include two or more different polymeric layers, with the innermost layer being formed of a lubricious polymer and/or including a lubricious coating.

A braid 104 is disposed between the outer polymeric layer 100 and the inner polymeric layer 102. The braid 104 may take any of a variety of forms. For example, the braid 104 may be a knitted braid or a woven braid. The braid 104 may be formed of any number and style of filaments, in any desired knitting pattern or weaving pattern. The braid 104 may be a 1×1 braid, a 2×2 braid, a 3×3 braid or a 4×4 braid, for example. The braid 104 may be formed from one or more filaments having a circular cross-sectional profile, as shown. The braid 104 may be formed from one or more filaments having any other cross-sectional profile. The braid 104 may be formed from one or more filaments having a flattened or even rectilinear cross-sectional profile, for example. The braid 104 may extend the length of the elongate shaft 12. The braid 104 may only extend a portion of the length of the elongate shaft 12, for example.

FIG. 17 is a cross-sectional view taken along the line 17-17 of FIG. 15, showing a cross-section through one wall of the inflatable balloon 16. The inflatable balloon 16 includes an outer polymeric layer 106 and an inner polymeric layer 108. In some instances, the outer polymeric layer 106 may be different from the outer polymeric layer 100 of the elongate shaft 12. In some instances, the outer polymeric layer 106 may be the same polymer as the outer polymeric layer 100. The inner polymeric layer 108 may be different from the inner polymeric layer 102 of the elongate shaft 12. In some instances, the inner polymeric layer 108 may be the same polymer as the inner polymeric layer 102.

The braid 104 extends between the outer polymeric layer 106 and the inner polymeric layer 108. In some instances, the braid 104 may be the same braid that extends through the elongate shaft 12. In other instances, the braid 104 may be a separate braid from the braid extending through the elongate shaft 12. The braid 104 may extend at least partially through the elongate shaft 12 and through the inflatable balloon 16 in order to provide additional tensile strength to the medical device 90. In some instances, the braid 104 may extend all of the way through the inflatable balloon 16. In some instances, the braid 104 may only extend partially through the inflatable balloon 16. As an example, the braid 104 may extend from the elongate shaft 12 to a midpoint of the inflatable balloon 16.

The medical device 90 includes the polymeric pad 48 that helps to secure the blade 18 to the inflatable balloon 16. In some instances, the blade 18 may be partially embedded within the polymeric pad 48. As an example, the blade 18 may include the T-shaped slots 56 (FIG. 3) that not only provide additional flexibility but also help to embed the blade 18 into the polymeric pad 48. The polymeric pad 48 may be formed of any of a number of different polymers.

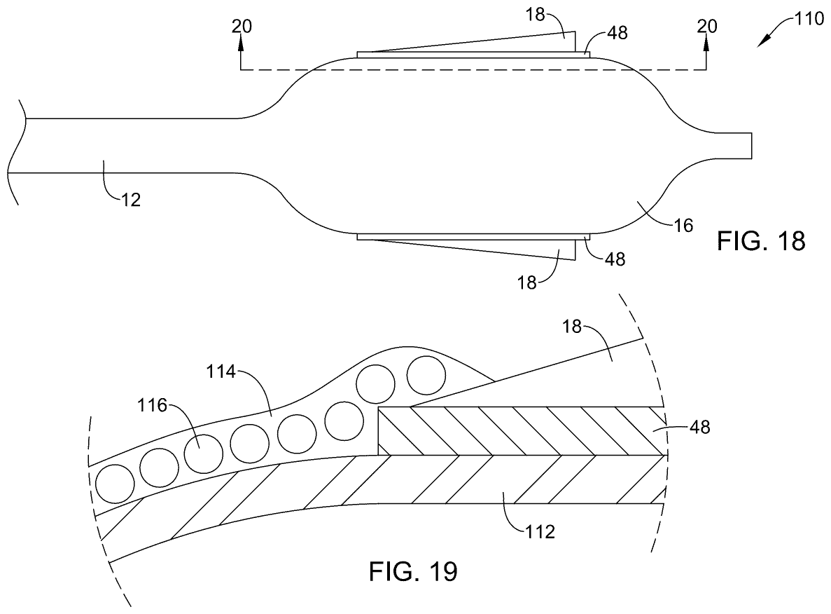

FIG. 18 is a schematic view of an example medical device 110. The example medical device 110 includes the inflatable balloon 16 secured to the elongate shaft 12. Two blades 18 are visible secured relative to the inflatable balloon 16. In some instances, there may be only one blade 18 secured relative to the inflatable balloon 16. In some instances, there may be three, four, five or more blades 18 secured relative to the inflatable balloon 16. In some instances, if there are two or more blades 18 secured relative to the inflatable balloon 16, the two or more blades 18 may be evenly or unevenly spaced circumferentially about the inflatable balloon 16. The blade or blades 18 are each secured to the inflatable balloon 16 via the polymeric pads 48.

In some instances, a braid may be used to help secure the blade or blades 18 in position. FIG. 19 is a cross-sectional view taken along the line 19-19 of FIG. 18. The inflatable balloon 16 includes a polymeric layer 112 to which the polymeric pad 48 is secured, thereby at least partially anchoring the blade 18 relative to the inflatable balloon 16. An outer layer 114, which may include a braid 116 that is embedded within a polymer, extends over the polymeric layer 112. The outer layer 114 also extends at least partially over the polymeric pad 48 and over a portion of the blade 18, thereby further securing the blade 18 relative to the inflatable balloon 16. In some instances, the outer layer 114 may only include the braid 116, and may not include a polymer in which the braid 116 is embedded. Thus, the braid 116 may extend radially outward of the base of the blade 18 and/or the polymeric pad 48 to further secure the blade 18 to the balloon 16, such that the base of the blade 18 and/or the polymeric pad 48 is interposed between the braid 116 and the polymeric layer 112 of the balloon 16.

The braid 116 may take any of a variety of forms. For example, the braid 116 may be a knitted braid or a woven braid. The braid 116 may be formed of any number and style of filaments, in any desired knitting pattern or weaving pattern. The braid 116 may be a 1×1 braid, a 2×2 braid, a 3×3 braid or a 4×4 braid, for example. The braid 116 may be formed from one or more filaments having a circular cross-sectional profile, as shown. The braid 116 may be formed from one or more filaments having any other cross-sectional profile. The braid 116 may be formed from one or more filaments having a flattened or even rectilinear cross-sectional profile, for example. The braid 116 may only extend over part of the inflatable balloon 16 itself. In some instances, the braid 116 may also extend along at least part of the length of the elongate shaft 12.

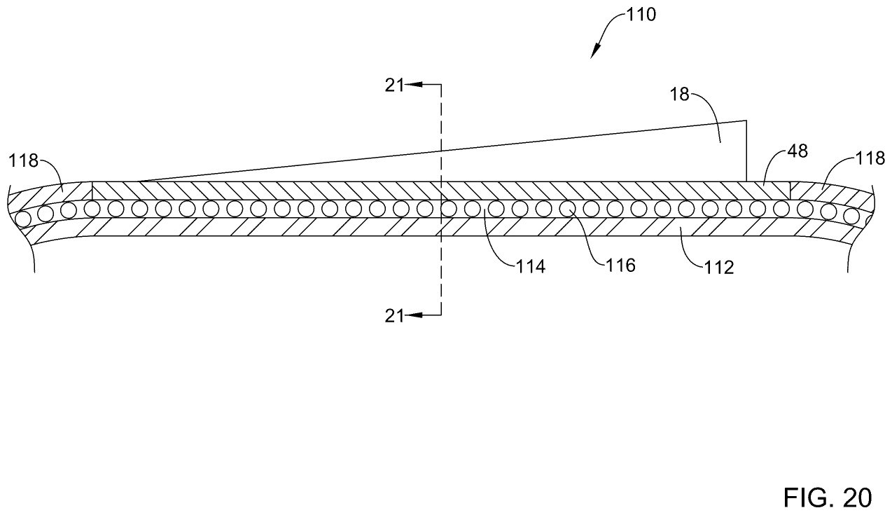

In some instances, the braid 116 may extend all of the way across the length of the inflatable balloon 16. In some instances, the braid 116 may be cut, formed or otherwise manipulated to cover at least the ends and/or base of the blade 18 (as shown in FIG. 19) while allowing the cutting edge of the blade 18 to otherwise extend above the braid 116. FIG. 20 is a cross-sectional view taken along the line 20-20 of FIG. 19, showing how the braid 116 (possibly embedded in a polymer to form the outer layer 114) extends along the inflatable balloon 16 the length (and possibly longer) of the blade 18. Thus, in some instances, the braid 116 may extend proximal of the proximal end of the blade 18 and/or may extend distal of the distal end of the blade 18. In some instances, the medical device 110 may include another polymeric layer 118 that covers the braid 116. As an example, the inflatable balloon 16 may be dip coated with a polymer such as a polyurethane to coat the braid 116, thereby forming the polymeric layer 118.

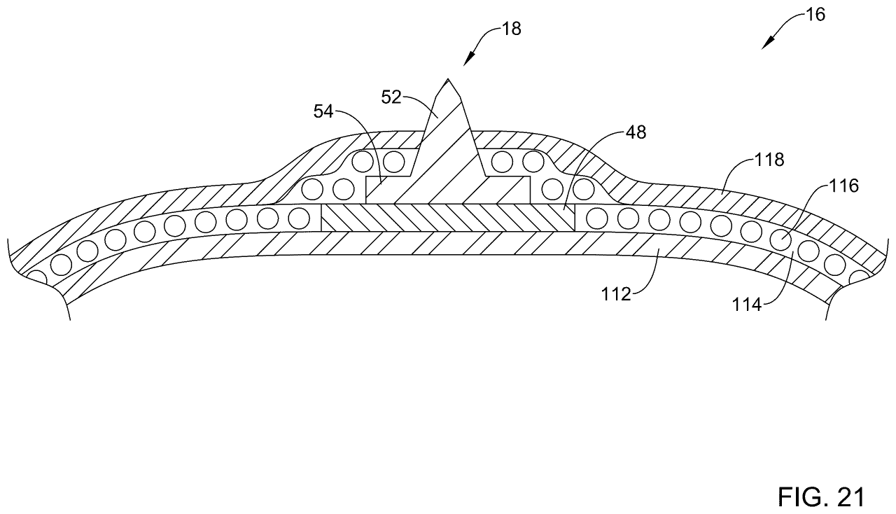

FIG. 21 is a cross-sectional view taken along the line 21-21 of FIG. 20. The blade 18 may be considered as including the base portion 54 that is embedded within the polymeric pad 48 and the blade portion 52 that extends outwardly from the base portion 54. As can be seen, the braid 116 extends over (i.e., radially outward of) at least the base portion 54 of the blade 18, thereby further securing the blade 18 in place relative to the inflatable balloon 16. The braid 116, which may itself form the layer 114, or may be embedded within a polymer to form the layer 114. In some instances, the assembly (e.g., the balloon 16 and blade 18 construct) may also be dip coated with a polymer such as a polyurethane to form the polymeric layer 118 subsequent to placing the braid 116 over the base portion 54 of the blade 18.

The materials that can be used for the various components of the medical devices and the various elements thereof disclosed herein may include those commonly associated with medical devices. In some embodiments, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some instances, the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device, comprising:
a catheter shaft including a distal region;
an inflatable balloon secured to the distal region of the catheter shaft, the inflatable balloon having an outer surface, the inflatable balloon defining a centerline when inflated; and
a blade extending over and secured to the outer surface, the blade having a cutting edge extending from a distal end of the blade to a proximal end of the blade, when the inflatable balloon is inflated, the cutting edge tapers from a maximum distance measured relative to a centerline of the balloon at the distal end of the blade to a minimum distance measured relative to the centerline of the balloon at the proximal end of the blade.

2. The medical device of claim 1, wherein the cutting edge of the blade comprises a straight line.

3. The medical device of claim 1, wherein the minimum distance is in a range of 1 to 2 millimeters and the maximum distance is in a range of 2 to 4 millimeters.

4. The medical device of claim 1, further comprising a braid extending through at least a portion of the elongate shaft and/or the inflatable balloon.

5. The medical device of claim 1, wherein when inflated, the outer surface of the inflatable balloon is a constant distance from the centerline of the balloon.

6. The medical device of claim 5, wherein the cutting edge of the blade tapers from the distal end of the blade to the proximal end of the blade.

7. The medical device of claim 6, wherein the cutting edge of the blade defines a blade height above the outer surface of the inflatable balloon, and the blade height tapers from a maximum blade height at the distal end of the blade to a minimum blade height at the proximal end of the blade.

8. The medical device of claim 7, wherein the maximum blade height is in a range of 1 to 2 millimeters.

9. The medical device of claim 8, wherein the minimum blade height is in a range of 0 to 0.5 millimeters.

10. The medical device of claim 1, wherein when inflated, the outer surface of the inflatable balloon is a varying distance relative to the centerline of the balloon.

11. The medical device of claim 10, wherein the distance between the outer surface of the inflatable balloon and the longitudinal axis is at a maximum at a distal region of the inflatable balloon and is at a minimum at a proximal region of the inflatable balloon.

12. The medical device of claim 11, wherein the cutting edge of the blade has a constant height relative to the outer surface of the inflatable balloon.

13. The medical device of claim 11, wherein the cutting edge of the blade has a varying height relative to the outer surface of the inflatable balloon.

14. The medical device of claim 1, further comprising one or more additional blades, each of the one or more additional blades extending over and secured to the outer surface, each of the one or more additional blades having a cutting edge extending from a distal end of the blade to a proximal end of the blade, the cutting edge extending when the inflatable balloon is inflated from a maximum distance measured relative to a centerline of the balloon at the distal end of the blade to a minimum distance measured relative to the centerline of the balloon at the proximal end of the blade.

15. A medical device, comprising:
a catheter shaft including a distal region;
an inflatable balloon secured to the distal region of the catheter shaft, the inflatable balloon including:
a constant diameter middle section corresponding to where the polymeric pad is secured to the outer surface of the inflatable balloon;
a distal region that tapers to a distal waist; and
a proximal region that tapers to a proximal waist;
a polymeric pad secured to the constant diameter middle section of the inflatable balloon; and
a blade secured within the polymeric pad, the blade defining a tapered cutting edge extending from a distal end of the blade to a proximal end of the blade, the tapered cutting edge having a maximum blade height relative to the polymeric pad at the distal end of the blade and a minimum blade height relative to the polymeric pad at the proximal end of the blade.

16. The medical device of claim 15, wherein the constant diameter middle section has a diameter when inflated in a range of 1.5 to 2.5 millimeters.

17. The medical device of claim 15, wherein:
the minimum blade height is in a range of 0 to 0.5 millimeters; and
the maximum blade height is in a range of 1 to 2 millimeters.

18. A medical device, comprising:
a catheter shaft including a distal region;
an inflatable balloon secured to the distal region of the catheter shaft, the inflatable balloon including a tapered middle section corresponding to where the polymeric pad is secured to the outer surface of the inflatable balloon, the tapered middle section tapering from a maximum diameter near a distal region to a minimum diameter near a proximal region;
a polymeric pad secured to the tapered middle section of the inflatable balloon; and
a blade secured within the polymeric pad, the blade defining a cutting edge extending from a distal end of the blade to a proximal end of the blade.

19. The medical device of claim 18, wherein the cutting edge has a uniform height measured relative to the polymeric pad.

20. The medical device of claim 18, wherein the cutting edge has a varying height measured relative to the polymeric pad.
